SEQUENCE LISTING (2 sequences)

SEQ ID NO: 1 - DNA, 20 bp, Artificial Sequence
  Synthetic
tcctccctgctccgattccg

SEQ ID NO: 2 - DNA, 23 bp, Artificial Sequence
  Synthetic
aggcaataggttttgagggccat

(19) United States
(12) Patent Application Publication
(10) Pub. No.: US 2003/0022191 A1
(43) Pub. Date: Jan. 30, 2003

(54) Method of identifying and managing increased risk of breast carcinoma associated with polymorphisms in MHC genes

(76) Inventor: Lotfi Chouchane, Monastir (TN)

Correspondence Address: Licata & Tyrrell P.C., 66 E. Main Street, Marlton, NJ, 08053, US

(21) Appl. No.: 10/040,707

(22) Filed: Jan. 7, 2002

Related U.S. Application Data
(60) Provisional application No. 60/260,242, filed on Jan. 8, 2001.

(51) Int. Cl. C12Q 1/68; C12P 19/34

(52) U.S. Cl. 435/6; 435/91.2

(57) ABSTRACT

This invention relates to a diagnostic methods of identifying an individual at an increased risk of breast carcinoma associated with a polymorphism in an MHC gene, by determining the genotypes of an individual and identifying polymorphisms associated with the predisposition or susceptibility to breast carcinoma. Also provided is a method of managing and treating patients with an increased risk or predisposition to breast carcinoma. The invention also provides to screening assays, and prophylactic and therapeutic methods discovered using such screening assays.

### INTRODUCTION

This application claims the benefit of priority from U.S. provisional application Serial No. 60/260,242 filed Jan. 8, 2001.

### FIELD OF THE INVENTION

This invention relates to diagnostic methods based upon a polymorphism in individuals indicative of an increased risk of breast carcinoma. More specifically, this invention relates to a method for diagnosis of an increased risk of breast carcinoma by screening for the presence of genetic polymorphisms in individuals, specifically the TNF-α and HSP70-2 genes. A method for predicting the probable survival of a patient with a polymorphism associated with breast carcinoma is also provided. This invention also relates to compositions for screening for the polymorphisms and improved treatment options for patients having identified polymorphisms.

### BACKGROUND OF THE INVENTION

Breast carcinoma represents a malignant proliferation of epithelial cells lining the ducts or lobules of the breast. In 1996 there were approximately 185,000 cases of invasive breast carcinoma and 46,000 deaths in the United States. Breast carcinoma is the most common carcinoma in women, with the exception of skin carcinoma. Human breast carcinoma is a clonal disease. A single transformed cell, the result of a somatic (acquired) or germline (inherited) mutation, becomes able to express full malignant potential in a series of events that occur in a sequential and stochastic manner. Thus, breast carcinoma is able to exist for a long time as a noninvasive disease, or an invasive but non-metastatic disease. This makes the need for timely diagnosis and appropriate management more urgent.

About 10 percent of the human breast carcinomas can be linked directly to germline mutations. This area has undergone remarkable evolution with the identification of several genes responsible for the familial cases. The first to be identified were germ line mutations in the tumor suppressor gene p53. In the disorder caused by these mutations inherited mutation in p53 lead to an increased risk of breast carcinoma and other malignancies.

Another putative tumor suppressor gene, BRCA-1, has been identified at the chromosomal locus 17q21; this gene encodes a zinc finger protein and the product therefore may function as a transcription factor. Women who inherit a mutated allele of this gene from either parent have an approximately 85 to 90 percent lifetime chance of developing breast carcinoma. Men who carry the mutant allele have an increased risk of prostate carcinoma, but not usually of breast carcinoma. Another gene termed BRCA-2, which has been localized to chromosome 11, is associated with an increased incidence of breast carcinoma in men and women.

The ataxia-telangiestasia gene is associated with remarkable radiation sensitivity even in a heterozygous state, which occurs in the population at a frequency of 1 to 2 percent. Because of susceptibility to radiation induced carcinoma, heterozygous carriers of this gene may be at risk from such procedures as screening mammograms.

Even more important than the roles of certain genes in inherited forms of breast carcinoma susceptibility may be their role in sporadic breast carcinoma. For example the p53 mutation is present in approximately 40 percent of human breast carcinomas as an acquired defect. In addition, as evidenced by loss of heterozygosity other types of tumor-suppressor activity appear to be lost in sporadic cases of human breast carcinoma. One dominant oncogene plays a role in about twenty-five percent of human breast carcinoma cases. The product of this gene a member of the EGF receptor superfamily called erbB2 (HER-2, neu) is overexpressed in these breast carcinomas owing to gene amplification and this overexpression can transform human breast epithelium.

Further, heat shock proteins (HSP), or stress proteins, are expressed in response to heat shock and a variety of other stress stimuli including oxidative free radicals and toxic metal ions. HSPs are involved in a number of protein complexes that involve cytokines, a group of molecules which includes TNF-α, which are important regulatory factors. Because HSPs are chaperone molecules they regulate DNA binding and DNA repair mechanisms which is an especially important somatic mutation mechanism for the occurrence of cancers. The human HSP70, or HSPA, a multigene family encodes several highly conserved 70-KD proteins with structural and functional properties in common, but which vary in their inducibility in response to metabolic stress. Sargent et al. (Proc. Natl. Acad. Sci. 1989.86:1968-72) identified a duplicated HSP70 locus in the class III region of the major histocompatibility complex on 6p21.3. These loci, HSP70-1 (HSPA1A; 14050) and HSP70-2 (HSPA1B) are 12 KB apart and lie 92 KB telomeric to the C2 gene. Milner and Campbell (Immunogenics 1990.32:242-51) determined that the HSP70-2 gene, like HSP70-1 lacks introns. The HSP70-1 and -2 coding sequences, which differ by 8 bp that do not alter the derived amino acid sequence, encode identical 641-amino acid proteins. The 3′ untranslated regions of these genes are completely divergent. Northern blot analysis of HeLa cell RNA detected an approximately 2.4 KB HSP70-2 transcript that was expressed at elevated levels following heat shock. Milner and Campbell, supra, investigated the presence of sequence variation in the HSP70-2 gene among different HLA haplotypes. They found only very limited sequence variation, which did not result in amino acid substitutions.

TNF, like HSP70-2, is located within the Major Histocompatibility Complex (MHC). Unraveling the importance of genetic variation in any of the MHC genes to disease susceptibility or severity is complicated by their location within the MHC, a highly polymorphic region that encodes numerous genes involved in immunologic responses. Both TNF and HSP70-2 are within the same MHC class, class IV, which is only 40-50 KB in total length.

Activated macrophages constitute the major cellular origin of TNF. TNF is associated with in vivo and in vitro killing of tumor cells (apoptosis and necrosis). It was originally discovered in the sera of mice and rabbits. Serum from such animals produced hemorrhagic necrosis and in some instances complete regression of certain transplanted tumors in mice.

The first bi-allelic TNFA polymorphism was detected in humans involving a single base change from G to A at position −308 in the promoter region of the gene. (Wilson et al. Human Mol. Genet. 1993.1:353-9). The less common allele A at −308 (called T2) shows an increased frequency in patients with Insulin Dependant Diabetes Melitus (IDDM), but this depends on the concurrent increase in HLADR3 with which T2 is associated. Disregulation and, in particular, overproduction of TNF have been implicated in a variety of human diseases, including sepsis, cerebral malaria, susceptibility to septic shock, and autoimmune diseases such as multiple sclerosis, rheumatoid arthritis, systemic lupus erythematosus, Crohn's disease, and in cachexima accompanying cancer and AIDS. Susceptibility to may of these diseases is thought to have a genetic basis and the TNF gene is considered a candidate predisposing gene.

The eventual hope for patients at an increased risk of breast carcinoma is to alter the course of the disease by directly targeting the genes responsible for the malignant process. The present invention provides a method to identify patients having an increased risk of breast carcinoma by the methods of the present invention thereby also providing effective treatment options for patients at an increased risk. It also provides a method for predicting the eventual clinical outcome of a breast carcinoma patient.

### BRIEF SUMMARY OF THE INVENTION

It is a particular object of this invention to provide a method of identifying an individual at an increased risk of breast carcinoma associated with a polymorphism in an MHC gene, comprising determining the genotypes of an individual and identifying polymorphisms in the individual associated with the predisposition or susceptibility to breast carcinoma. Polymorphisms of particular interest are on the TNF-α gene at the −308 locus, and on the HSP70-2 gene at the 1267 locus.

Also provided is a method of managing and treating patients with an increased risk or predisposition to breast carcinoma. The invention also relates to screening assays, and prophylactic and therapeutic methods discovered using such screening assays. Also related is a method of predicting the clinical outcome of a breast carcinoma patient comprising determining the individual's genotype and the rate of survival associated with the genotype.

### DETAILED DESCRIPTION

In a first aspect, the invention provides a method of identifying an individual at an increased risk of breast carcinoma associated with a polymorphism in an MHC gene, comprising determining the genotype of an individual and identifying polymorphisms in an MHC gene associated with the predisposition or susceptibility to breast carcinoma.

In a preferred embodiment of the invention the method is to screen for an individual at risk of a condition or disease such as an increased risk of breast carcinoma by identifying polymorphisms in TNF-α at −308 and in HSP70-2 at −1267.

The invention is based upon a correlation between polymorphisms in the TNF-α gene and HSP70-2 genes, (specifically at position −308 and 1267 respectfully), and an increased risk of breast carcinoma. The invention is of advantage in that by screening for the presence polymorphisms it is possible to identify individuals likely to have a genetic predisposition or susceptibility to such increased risk. It may also result in substantially different management, especially prevention and treatment (vaccination).

In one embodiment of the invention, diagnosis is carried out by determining whether an individual carries a polymorphism associated with breast carcinoma. Genotypic and allelic frequencies of this invention are readily determined by a number of methods known to those skilled in the art. Examples are provided herein and include using PCR amplification and restriction enzyme digestion.

In a preferred embodiment of the invention is provided a method of identifying a predisposition or susceptibility to breast carcinoma, comprising determining whether the individual possesses a polymorphic risk version of the TNF-α gene, a polymorphic risk version of the TNF-α gene being one that has an A at site at the −308 site, the method comprises digestion of corresponding PCR products with the endonuclease Nco I, analysis of amplified fragments by agarose-gel electrophoresis, wherein the presence of Nco I site is indicated by the cleavage of the 107 bp amplified fragment to yield fragments of 87 bp and 20 bp, and wherein the two allelic forms of TNF-α corresponding to the presence or absence of NcoI are referred to as TNF-1 and TNF-2 respectively; and identification of the presence of susceptibility to breast carcinomas greatest if that individual is homozygous for the polymorphic risk version of the gene at the −308 site (TNF2/TNF2).

In another preferred embodiment of the invention is provided a method of identifying a predisposition or susceptibility to breast carcinoma, the method comprising determining whether the individual possesses a polymorphic risk version of the HSP70-2 gene. A polymorphic risk version of the HSP70-2 gene has been determined to be the homozygous genotype (P2/P2 or A/A) which has been found to lack a Pst I site at the 1267 position. The Pst I polymorphism in HSP70-2 gene is a G to A polymorphism. The method comprises digestion of corresponding PCR products, analysis of amplified fragments by agarose-gel electrophoresis, wherein the presence of the Pst I site is indicated by the cleavage of the 2075 bp amplified product to yield fragments of 1139 bp and 936 bp, and wherein the two allelic forms of HSP70-2 gene corresponding to the presence or absence of Pst I site are referred to as HSPP1 and HSPP2 respectively, and identification of the presence of susceptibility to breast carcinomas greatest if that individual is homozygous for the polymorphic risk version of the gene at the 1267 site (P2/P2). Multiple techniques exist and are known to one skilled in the art in the manufacture of means for diagnosing whether an individual has an increased risk of breast carcinoma, by determining the genotype of the MHC gene e.g., TNF-α having an A at the −308 site or the HSP70-2 gene lacking a Pst I site. One can use restriction analysis which generates different fragment lengths for the differing allele types and then identify by electrophoresis on an agarose gel where the different fragments migrate based on size.

The methods conveniently comprise amplifying fragments of the TNF-α and the HSP70-2 genes to produce copies and determining whether copies of the fragments contain the particular genotypes associated with cancer.

Another suitable technique is to amplify the fragment using PCR techniques, producing copies of a fragment that is at least 500 base pairs in length, preferably at least 600 base pairs in length. It is preferred that the PCR primers are selected so as to amplify a region of the gene that is about 740 base pairs in length. PCR techniques are well known in the art and it would be within the ambit of a person of ordinary skill in this art to identify primers for amplifying a suitable section of the applicable exon of the TNF-α gene. PCR techniques are described for example in EP-A-0200362 and EP-A-0201 184. In a further embodiment of the invention, the diagnostic method comprises analysis of the polymorphisms using single strand conformational polymorphism (SSCP) mapping to determine whether the genes are the risk or the non-risk forms, i.e., the G to A transition at the −308 site of the TNF-α , the G to A transition for the 1267 site of the HSP70-2. As described above, in preferred embodiments of the invention, the method comprises screening for polymorphisms associated with breast carcinoma, and this screening is conveniently carried out by any one of a number of suitable techniques that are known in the art, and may be conveniently selected from amplification of a nucleic acid sequence located within an MHC gene. Southern blotting of regions of the gene and single strand conformational polymorphism mapping of regions within the gene or as described in the example below. The genotype in that region is also optionally determined using a variety of methods including hybridization probes adapted selectively to hybridize with the particular polymorphisms of an MHC gene which are associated with predisposition or susceptibility to disease. A probe used for hybridization detection methods must be in some way labeled so as to enable detection of successfully hybridization events. This is optionally achieved by in vitro methods such as nick-translation, replacing nucleotides in the probe by radioactively labeled nucleotides, or by random primer extension, in which non-labeled molecules act as a template for the synthesis of labeled copies. Other standard method of labeling probes so as to detect hybridization are known to those skilled in this art.

According to another aspect of the invention there is provided a method of diagnosis and therapy comprising diagnosing patients at increased risk of breast carcinoma by identifying a polymorphism in an MHC gene and treating an individual having such increased risk by methods known to those of skill in the art. It is preferable to do so prior to the patient having breast carcinoma. Breast carcinoma can be diagnosed by methods known to those of skill in the art and as described herein.

In a preferred embodiment of this invention the polymorphism is located on the TNF-α gene, and the polymorphic risk version of the TNF-α gene has a G to A transition polymorphism at position −308. In another preferred embodiment, the polymorphism is located on the HSP70-2 gene, and the polymorphic risk version of the HSP70-2 gene lacks a Pst I site at position 1267.

Known therapies for breast carcinoma may be effective in halting advancement of the disease, or at least slowing the advancement. TNF-α −308 and HSP 70-2 gene analysis in accordance with the teachings of the invention may also lead to more appropriate preventative measures, such as vaccination, and placement of patients into intensive care/critical care units, an important factor in optimizing survival from breast carcinoma. It is thus an advantage of the invention that early identification of patients at increased risk of breast carcinoma is improved, so that preventative therapy can be started as soon as possible, optimizing any interventions potential (such as vaccination and immunomodulatory therapy) for affecting outcome. The decision of a physician on how and whether to initiate therapy in anticipation of the disease can be taken with increased confidence.

Another aspect of the invention provides a composition for use in diagnosing a disease associated with a genetic polymorphism associated with breast carcinoma in an MHC gene in an individual predisposed or susceptible to said increased risk of breast carcinoma, said composition comprising one or more primer nucleic acid molecules adapted to amplify portions of the MHC gene.

For example, the composition may comprise a nucleic acid molecule capable of identifying the G to A transition at the −308 site in the TNF-α gene, said genotype being indicative of a risk genotype in said individual. It may also comprise singularly or in combination a nucleic acid molecule capable of identifying the polymorphism in the HSP70-2 gene (P2/P2), said genotype being indicative of a risk genotype in said individual

A further embodiment of this aspect of the invention provides a composition for identifying individuals at increased risk of breast carcinoma comprising means for determining the genotype in an MHC gene. In a preferred embodiment of the invention, a composition comprises PCR primers adapted to amplify a DNA sequence within and around the TNF-α−308 location and/or the HSP70-2 −1267 location, wherein alternative versions of the gene are distinguished one from another.

In a further aspect of the invention there is provided a kit comprising a diagnostic composition such as described above and an indicator composition for indicating how possessing an MHC genotype correlates with having an increased risk of breast carcinoma. Diagnostic kits are typically accompanied by or comprise a chart or other visual aid for assistance in interpreting the results obtained using the kit. Suitable indicator compositions for use in the diagnostic kit of the invention include a leaflet or other visual reminder that possessing the risk polymorphism version of the MHC gene correlates with increased risk of breast carcinoma.

In a still further aspect of the invention there is provided use, in the manufacture of means for diagnosing whether an individual has an increased risk of breast carcinoma, of PCR primers adapted to amplify a region in an MHC gene. Alternative versions of the genes are typically distinguished one from another by means known to those skilled in the art.

Multiple techniques exists and are known to one skilled in the art in the manufacture of means for diagnosing whether an individual has an increased risk of breast carcinoma and the survival rate by determining the polymorphisms associated with breast carcinoma in an MHC gene. One can use restriction analysis which generates different fragment lengths for the A allele (GA and GG genotype), identified by electrophoresis on an agarose gel where the different fragments migrate different amounts based on their size.

Optionally, the assessment of an individual's risk factor according to any aspect of the invention is calculated by determining the presence of polymorphisms associated with breast carcinoma in an individual, and combining the result with analysis of other known genetic or physiological or other risk factors known to those of skill in the art. The invention in this way provides further information on which measurement of an individual's risk can be based.

In another embodiment of the invention, the results of the genotyping done herein are used, along with other diagnostics measures and disease parameters, by treatment providers to determine the best course of treatment for the patient having been determined as susceptible to increased risk of breast carcinoma by the methods of this invention.

The polymorphisms shown in polypeptides described in the present invention may be beneficially employed in a screening process for compounds which stimulate (agonists) or inhibit (antagonists, or otherwise called inhibitors) the synthesis or action of the TNF-α polypeptide. The polypeptides may also be employed in a screening process for compounds which mimic the agonist or antagonist properties of the polypeptides. Thus, the polypeptides encoded by may also be used to assess and identify agonist or antagonists from, for example, cells, cell-free preparations, chemical libraries, and natural product mixtures. These agonists or antagonists may be natural substrates, ligands, receptors, etc., as the case may be, of the polypeptide of the present invention; or may be structural or functional mimetics of the polypeptide of the present invention. See Coligan et al. Current Protocols in Immunology 1(2):Chapter 5 (1991).

TNF-α proteins and heat shock proteins are ubiquitous in the mammalian host, including humans, and are responsible for many biological functions, including many pathologies. Accordingly, it is desirous to find compounds and drugs which are able to both stimulate these polypeptides on the one hand and which can also inhibit the function of such polypeptides on the other hand.

In general, such screening procedures may involve identifying, generating and using appropriate cells which express the receptor of the TNF-α and HSP70-2 polypeptides on the surface thereof. Such cells include cells from mammals, yeast, Drosophila or E. coli. Such cells may be identified, for example, by direct binding methods using radiolabeled or fluorescently tagged TNF-α or HSP70-2 polypeptides. Cells expressing these polypeptide receptors (or cell membrane containing the expressed polypeptides) are then contacted with a test compound to observe binding, or stimulation or inhibition of a functional response. Alternatively, the cDNA for polypeptide receptors may be cloned by the above direct binding methods using expression cloning or purification methods known in the art, and its extracellular domain expressed as a secreted or membrane protein. The soluble or membrane bound receptor can then be used to identify agonists or antagonists via direct binding methods.

The assays may simply test binding of a candidate compound wherein adherence to the cells bearing the polypeptide receptor is detected by means of a label directly or indirectly associated with the candidate compound or in an assay involving competition with a labeled polypeptide. Further, these assays may test whether the candidate compound results in a signal similar to that generated by binding of the appropriate polypeptide, using detection systems appropriate to the cells bearing the polypeptide receptors at their surfaces. Inhibitors of activation are generally assayed in the presence of a known agonist and the effect on activation by the agonist by the presence of the candidate compound is observed. Standard methods for conducting such screening assays are well understood in the art.

Examples of potential polypeptide antagonists include antibodies or, in some cases, oligonucleotides or proteins which are closely related to the ligands, substrates, receptors, etc., as the case may be, of the polypeptide, e.g., a fragment of the ligands, substrates, receptors, or small molecules which bind to the target receptor of the present invention but do not elicit a response, so that the activity of the polypeptide is prevented. Preferred are those that can access and affect cellular function.

This invention provides methods of treating an abnormal conditions related to both an excess of and insufficient amounts of polypeptide activity.

If the activity of the polypeptide is in excess, several approaches are available. One approach comprises administering to a subject an inhibitor compound (antagonist) as herein above described along with a pharmaceutically acceptable carrier in an amount effective to inhibit activation by blocking binding of the polypeptide to its target receptor, or by inhibiting a second signal, and thereby alleviating the abnormal condition, i.e., increased risk of breast carcinoma.

In another approach, soluble forms of the polypeptides capable of binding its receptor in competition with endogenous polypeptide may be administered. Typical embodiments of such competitors comprise fragments of the polypeptide.

In still another approach, expression of the MHC gene encoding endogenous polypeptides can be inhibited using expression blocking techniques. Known such techniques involve the use of antisense sequences, either internally generated or separately administered. See, for example, O'Connor, J. Neurochem. 1991.56:560 in Oligodeoxynucleotides as Antisense Inhibitors of Gene Expression, CRC Press, Boca Raton, Fla. (1988). Alternatively, oligonucleotides which form triple helices with the gene can be supplied. See, for example, Lee et al. Nucleic Acids Res. 1979.6:3073; Cooney et al. Science 1988.241:456; Dervan et al. Science 1991.251:1360. These oligomers can be administered per se or the relevant oligomers can be expressed in vivo.

For treating abnormal conditions related to an under-expression of polypeptide activity several approaches are also available. One approach comprises administering to a subject a therapeutically effective amount of the appropriate polypeptide or a compound, i.e., an agonist or mimetic as described above, in combination with a pharmaceutically acceptable carrier, to thereby alleviate the abnormal condition. Alternatively, gene therapy may be employed to effect the endogenous production of TNF-α by the relevant cells in the subject. For example, a polynucleotide of the invention may be engineered for expression in a replication defective retroviral vector, as discussed above. The retroviral expression construct may then be isolated and introduced into a packaging cell transduced with a retroviral plasmid vector containing RNA encoding a polypeptide of the present invention such that the packaging cell now produces infectious viral particles containing the gene of interest. These producer cells may be administered to a subject for engineering cells in vivo and expression of the polypeptide in vivo. For overview of gene therapy, see Chapter 20, Gene Therapy and other Molecular Genetic-based Therapeutic Approaches, (and references cited therein) in Human Molecular Genetics, T. Strachan and A. P. Read, BIOS Scientific Publishers Ltd. (1996).

All such agonists and antagonists are administered in an amounts effective to treat the condition and in pharmaceutically acceptable carriers. Techniques for determining effective amounts and carriers are well known to those of skill in the art.

Another aspect of this invention is a method of predicting the clinical outcome of a breast carcinoma patient comprising determining the MHC genotype of an individual. For example, the homozygous (AA) genotype of the TNF-α gene at the −308 locus shows the TNF-breast carcinoma-specific overall survival and disease-free survival to be shortest in amongst the patients carrying this TNF2 homozygous genotype,and the longest amongst individuals possessing the HSP70-2 homozygous (AA) genotype.

The −308 TNF-α polymorphism and that of HSP70 genes were analyzed in a cohort study of patients with different malignant tumors. A higher relative frequency of the TNF2 allele was shown in patients compared to controls. Independent from the TNF2 allele, polymorphism in HSP70 genes was found to be associated with malignant tumors.

Table 1 shows genotype frequencies for TNF-α and HSP70-2 in patients with breast carcinomaand the control group. A significant increase in the TNF2/TNF2 genotype was observed in the patient group. The relative risk (RR) of breast carcinomaassociated with the TNF2/TNF2 genotype was 4.44 (P-0.006).

The allelic frequency of the HSPP2 was 0.533 in patients with breast carcinomaand 0.425 in control subjects (P=0.002). The frequency of P1/P2 heterozygotes was 0.506 in the patient group and 0.747 in the control population (P=0.0001) resulting in a significantly negative RR associated with this genotype. Conversely, the frequency of the P2/P2 homozygotes was 0.280 in the patient group and only 0.052 in controls. These results indicate that the RR of breast carcinoma associated with the HSP70-2 polymorphism is confined to P2/P2 homozygotes (RR=7.12, P=0.0001).

TABLE 1
TNF-α and HSP70-2 Genotype Frequencies in Control
Subjects adn in Patients with Breast Carcinoma

| | | Breast | Breast |
|---|---|---|---|
| | Controls | carcinoma | carcinoma |
| | n = 174 | (n = 243) | (n = 243) |
| Genotype | f | f | P value |
| TNF-α | | | |
| TNF1/TNF1 | 0.672 | 0.687 | NS |
| (G/G) | | | |
| TNF1/TNF2 | 0.305 | 0.218 | NS |
| (G/A) | | | |
| TNF2/TNF2 | 0.023 | 0.095 | 0.006 |
| (A/A) | | | |
| HSP70-2 | | | |
| P1/P1 | 0.201 | 0.214 | NS |
| (G/G) | | | |
| P1/P2 | 0.747 | 0.506 | 0.0001 |
| (G/A) | | | |
| P2/P2 | 0.052 | 0.280 | 0.0001 |
| (A/A) | | | |

The chi-square test with Yates' correction was used to determine whether significant differences (P value) were observed when patient group was compared with control subjects. NS=not significant; f=frequencies.

Another aspect of this invention provides a method of predicting the clinical outcome (considered to be the 6-year breast carcinoma-specific overall survival rates of a breast carcinoma patient) comprising determining whether the individual possesses a TNF2 homozygous genotype of the TNF-α gene at the −308 locus wherein the TNF-breast carcinoma-specific overall survival and disease-free survival are considered to be shortest in patients carrying the TNF2 homozygous genotype. Table 2 shows the clinicopathological characterization. A highly significant association was found between TNF2 homozygous genotype and breast carcinoma (RR=4.44, P=0.006. A high relative risk of breast carcinoma was found associated with one HSP70-2 homozygous genotype (P2/P2) (RR=7.12, P=0.0001).

TABLE 2
Clinicopathological Characteristics of the
243 Breast Carcinoma and the corresponding Univariate
analysis of death (OVS) and relapse (DFS).

| | | | | |
|---|---|---|---|---|
| | Breast | | | |
| | carcinoma | Breast | | |
| | specific | carcinoma | | |
| | OVS | specific | DFS | |
| Percent | 6 year | OVS | 6 year | DFS |
| (%) | rate | P value | rate | P value |

Clinical
tumor
size

| | | | | | |
|---|---|---|---|---|---|
| T1-T2 | 59.8 | 87.5 | <0.02 | 68.0 | <0.01 |
| T3-T4 | 40.2 | 68.0 | | 27.8 | |
| Lymph | | | | | |
| node | | | | | |
| status | | | | | |
| N (−) | 50.6 | 90.3 | <0.01 | 61.0 | <0.02 |
| N (+) | 49.4 | 69.4 | | 50.0 | |
| SBR | | | | | |
| grading | | | | | |
| 1-2 | 61.2 | 87.3 | <0.01 | 59.7 | <0.05 |
| 3 | 38.8 | 58.3 | | 36.1 | |
| Age | | | | | |
| <50 | 62.4 | 79.1 | NS | 61.1 | NS |
| year | | | | | |
| ≧50 | 37.6 | 80.5 | | 52.8 | |
| year | | | | | |

Six-year survival rates were estimated according to Kaplan and Meier. The log-rank test was used to determine whether significant differences (P value) were observed between subgroups of patients. The lymph node status was determined based on the pathological examination. NS=not significant.

The TNF2 homozygous genotype showed a significant association with reduced disease-free survival (DFS) and/or overall survival (OVS) by univariate test. Conversely, P2-HSP70-2 homozygous genotype associated with increased overall survival but not with disease-free survival. Multivariate analysis retained significance for TNF2 homozygous genotype as an independent prognostic indicator for both disease-free survival (RR=2.75, P=0.01) and overall survival (RR=4.08, P=0.01). DFS and breast carcinoma-specific OVS rates were estimated and compared by univariate analysis on these clinicopathological parameters. Significant associations were found for clinical tumor size, nodal status, and tumor grading with DFS and OVS. No significant differences were observed for age.

The breast carcinoma-specific overall survival and disease-free survival were significantly shorter in the group of patients carrying the TNF2 homozygous genotype. The estimated 3-year and 6-year breast carcinoma-specific overall survival rates in the groups of patients carrying or not carrying the TNF2 homozygous genotype were, respectively, 57% and 14% versus 91% and 89% (log-rank test, P<10⁻⁶). The 3-year DFS rate in the group of patients with TNF2/TNF2 genotype was 44.4% and 76% in that of patients without TNF2/TNF2 (log-rank test, P<10⁻³). The breast carcinoma-specific OVS was significantly longer in the group of patients carrying the HSP-P2 homozygous genotype. The estimated 6-year OVS rate in the groups of patients with HSP-P2 homozygous genotype was 96% and 76% in that of patients without HSP-P2/P2 marker (log-rank test, P<0.04). No statistical difference in disease-free survival was observed between the two groups of patients.

Multivariate analyses were undertaken to evaluate the importance of the TNF and HSP70-2 markers in the risk of relapse and death compared to the clinicopathological parameters. Introducing the genetic and the clinicopathological parameters bearing prognostic significance tested the Cox model.

The TNF2 homozygous genotype was found to be an independent risk factor for both disease-free survival (RR=2.75, P=0.01) and breast carcinoma-specific overall survival (RR=4.08, P=0.01). The tumor size was selected as an independent prognostic indicator for both DFS (RR=1.51, P=0.01) and OVS (RR=1.60, P=0.05). The SBR grade was retained as an independent risk factor for OVS (RR=2.90, P=0.04) but not for DFS. The nodal status and the HSP-P2 homozygous genotype were not selected for OVS and DFS.

The frequency of the TNF2 allele was found higher in patients group compared to controls but the difference in the allele frequency did not reach statistical significance. This result along with those showing that the −308 bp polymorphism at the TNF-α gene is a functionally important element influencing TNF-α production, and that the genetic basis of the high circulating TNF-α levels found in patients with carcinoma results from TNF-polymorphism. Prognostic significance evaluation of the TNF-α genetic marker in breast carcinoma indicated that TNF2 homozygous genotype is an independent risk factor of relapse and death. It is believed that individuals, who are genetically predisposed to increased TNF-α production, are at higher risk for chronic immune activation upon tumor challenge, yielding to several systemic symptoms, such as cachexia, anemia and poor performance status. All of these adverse conditions affect the ability of the host to tolerate treatment and consequently preclude disease's poor outcome. The increased endogenous TNF production by tumor cells could contribute to the resistance to chemotherapy.

The allele and genotype frequencies of HSP70-2 determined for the present cohort were similar to that of a previous study. Comparison of HSP70-2 allele and genotype frequencies in patients with breast carcinomaand control subjects indicated a decrease of P1/P2 genotype in the group of patients. Conversely, a high RR of breast carcinoma was associated the P2 homozygous genotype. Although the possible functional implications of HSP70 polymorphisms have not been studied, several features suggest that they may be among factors dictating the function of HSP70.

Over-expression of stress proteins in tumor cells is caused by the increased demand of accelerated cell proliferation and the harmful environment. Several reports highlighted the role of stress proteins in carcinoma pathogenesis and the disease progression. It has been shown that stress proteins including HSP70, participate in the folding of numerous proto-oncogene and oncogene products. Induction or over expression of various stress proteins protects host cells from apoptosis. Furthermore, HSP70 has been found to protect tumor cells from TNF-mediated cytotoxicity. In several malignant cell types the simultaneous induction of various stress proteins and multidrug resistance has been observed. Administration of chemotherapeutic agents leads to an increase in the expression of stress proteins in particular HSP70. Over-expression of HSP70 on tumor cells was correlated to poor prognosis in breast carcinoma.

The present invention shows the association between the HSP70-2 polymorphism and susceptibility and overall survival to breast carcinoma. It also demonstrates that the genetic basis of the various roles of HSP70 in tumor development and progression may result from HSP70-2 polymorphism. Polymorphisms in TNF-α and HSP70-2 represent attractive susceptibility markers for breast carcinoma.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following example, which is included herewith for purposes of illustration only and is not intended to be limiting of the invention.

### EXAMPLE 1

The gene and allele frequencies of the TNF-α and HSP70-2 genes were determined in a group of 174 control subjects and 243 patients with breast carcinoma. Controls and patients were selected from the same population living in the middle coast of Tunisia. Both control and patients groups include unrelated subjects.

Clinical follow-up data were collected on the cohort of the 243 patients recruited from the department of Radiation Oncology and Medical Oncology of Sousse Hospital, Sousse Tunisia, between 1991 and 1999. All patients included in this study had primary breast carcinoma, with unilateral breast tumors. The patients (239 females and 4 males) had a mean age of 48±11 years. The median of follow-up was 36 months (range, 1 to 120 months). At time of analysis, 57 patients relapsed (local or distant recurrence). Among them, 21 patients died from breast carcinoma (36.8%). Control subjects (75 females and 99 males) having a mean age of 39±12 years, were healthy blood donors having no evidence of any personal or family history of carcinoma (or other serious illness).

Among the 243 patients, 180 had surgery in association with additional therapy: radiotherapy alone was given to 16 patients, chemotherapy (generally six courses of 5-fluorouracil, Adriamycin, and cyclophosphamide) alone to 28 patients, both radiotherapy and chemotherapy to 103 patients, both radiotherapy and endocrine therapy (tamoxifen) to 4 patients, both chemotherapy and endocrine therapy to 6 patients and radiotherapy in association with chemotherapy and endocrine therapy to 23 patients.

Among the 63 patients who had not surgery: 16 patients had only radiotherapy, 17 had only chemotherapy, 23 had both radiotherapy and chemotherapy and 7 had chemotherapy in association with endocrine therapy.

Genomic DNA was extracted from peripheral blood leukocytes by a salting out procedure. Briefly, 5 ml of blood was mixed with Triton lysis buffer (0.32 M sucrose, 1% Triton X-100, 5 mM MgCl₂, H₂O, 10 mM Tris-HCl, pH 7.5). Leukocytes were spun down and washed with H₂O. The pellet was incubated with proteinase K at 56° C. and subsequently salted out at 4° C. using a saturated NaCl solution. Precipitated proteins were removed by centrifugation. The DNA in the supernatant was precipitated with ethanol. The DNA pellet was dissolved in 400 μl H₂O.

Based upon the method described by Cabrera et al. in J. Exp. Med. 1995.182:1259-64, a polymerase chain reaction followed by digestion with the endonuclease Nco I was used to detect the G to A transition polymorphism at position −308 of TNF-α gene. Two sequence specific oligonucleotide primers were used for the PCR: the 3′ primer (5′-TCCTCCCTGCTCCGATTCCG -3′; SEQ. ID NO:1) was used in combination with the 5′ primer (5′-AGGCAATAGGTTTTGAGGGCCAT-3′; SEQ. ID NO:2). Thirty microliters of PCR reaction mixture were comprised of genomic DNA samples (100 ng), 200 μmol/L dNTPs, 1.5 mM MgCl₂, 1×Taq polymerase buffer, 50 μmol of each primer and 0.5 unit of Taq DNA polymerase (Amersham, Paris, France). Reaction conditions used with the thermal cycler (Biometra, Gbttingem, Germany) were as follows: 95° C. for 5 minutes; 29 cycles of 95° C. for 30 seconds, 60° C. for 30 seconds and 72° C. for 45 seconds; 72° C. for 10 minutes. The PCR product (107 bp) was verified by DNA sequencing.

The amplified fragments (107 bp) were digested with Nco I and analyzed by agarose-gel electrophoresis. The presence of a Nco I site was indicated by the cleavage of the 107 bp amplified product to yield fragments of 87 bp and 20 bp. The two allelic forms of TNF-α, corresponding to the presence or the absence of the Nco I site, are referred to as TNF1 and TNF2 respectively.

Polymorphism within HSP70-2 gene has been characterized by Milner et al., who identified a polymorphic Pst I site at position 1267 of the HSP70-2 gene, see Immunogenetics 1992.36:357-62. The position 1267 of the HSP70-2 gene lies in the coding region. The coding sequence of the HSP70-2 gene was amplified from genomic DNA using sequence specific oligonucleotide primers: the 5′-primer: 5′-TCCGAAGGACTGAGCTCTTG-3′ (SEQ. ID NO:1) was used in combination with the 3′-primer: 5′-CAGCAAAGTCCTTGAGTCCC-3′ (SEQ. ID NO:2). The PCR reaction mixture contained 500 ng of genomic DNA; 200 μmol/L dNTPs; 1.5 mM MgCl₂; 1×Taq DNA polymerase buffer; 1 μmol each primer; and 1 unit of Taq DNA polymerase (Amersham, France). Amplification was accomplished by initial incubation at 94° C. for 5 min followed by 30 cycles of incubation at 94° C. for 1 min; 60° C. for 1 min; and 72° C. for 3 min, followed by a final incubation at 72° C. for 10 min.

To assess the polymorphism of the HSP70-2 at position 1267, the corresponding PCR products were digested with Pst I. The presence of an Pst I site was indicated by the cleavage of the 2075 bp amplified product to yield fragments of 1139 bp and 936 bp. The two allelic forms of HSP70-2, corresponding to the presence or the absence of the Pst I site, are referred to as HSPP1 and HSPP2 respectively.

The chi-square test with Yates' correction was used to test for significant association between disease (breast carcinomaagainst controls) and TNF-α or HSP70-2 alleles or genotypes. Relative risk of associated with a particular genotype was estimated by the “Odds Ratio” formula (OR) formula: OR=n1.n4/n2.n3, in which n1 is the proportion of patients with the genotype, n2 is the proportion of controls with genotype, and n3 and n4 are the corresponding proportions of individuals in patient and control groups without the genotype. OR was tested using a chi-square distribution, and the null hypothesis being tested was OR=1.

Disease-free survival (DFS) was defined as the time from the date of diagnosis to the first local or distant recurrence or to last contact. Breast carcinoma-specific overall survival (OVS) was defined as the time from the date of diagnosis to death if the patient died from breast carcinomaor to last contact. Six-year survival rates were estimated.

In multivariate analysis, relative risk of recurrence or death from breast carcinoma, 95% confidence intervals, and P values for censored survival data were calculated by use of COX's proportional hazards regression model Biometrics. 1982; 38: 541-61. All P calculations were two-sided and P was considered significant at <0.05. Only clinicopathological parameters bearing prognostic significance were included in the Cox model. Clinicopathological parameters were dichotomized as follows: nodal status (1 versus no positive lymph node), SBR (Scarff, Bloom and Richardson) tumor grade (1-2 versus 3), clinical tumor size (T₁-T₂ versus T₃-T₄)

What is claimed:

1. A method of identifying an individual at an increased risk of breast carcinoma associated with a polymorphism in a gene, comprising determining the MHC genotype of an individual and identifying polymorphisms associated with the predisposition or susceptibility to breast carcinoma.

2. The method of claim 1 wherein the polymorphism is on the TNF-α gene at the −308 locus.

3. The method of claim 1 wherein the polymorphism is on the HSP70-2 gene at the 1267 locus.

4. A method of identifying a predisposition or susceptibility to breast carcinoma, the method comprising determining whether the individual possesses a polymorphic risk version of the TNF-α gene, wherein the risk version has an A at site at the −308 site, the method comprising:
(a) digestion of corresponding PCR products with the endonuclease Nco I;
(b) analysis of amplified fragments by agarose-gel electrophoresis, wherein the presence of Nco I site is indicated y the cleavage of the 107 bp amplified fragment to yield fragments of 87 bp and 20 bp, and wherein the two allelic forms of TNF-α corresponding to the presence or absence of NcoI are referred to as TNF-1 and TNF-2 respectively;
(c) identifying the presence of susceptibility to breast carcinomas greatest if that individual is homozygous for the polymorphic risk version of the gene at the −308 site (TNF2/TNF2).

5. A method of identifying a predisposition or susceptibility to breast carcinoma, the method comprising determining whether the individual possesses a polymorphic risk version of the HSP70-2 gene, wherein the risk version of the HSP70-2 gene lacks a Pst I site at the 1267 position, the method comprising:
(a) digestion of corresponding PCR products with Pst I;
(b) analysis of amplified fragments by agarose-gel electrophoresis, wherein the presence of the Pst I site is indicated by the cleavage of the 2075 bp amplified product to yield fragments of 1139 bp and 936 bp, and wherein the two allelic forms of HSP70-2 gene corresponding to the presence or absence of Pst I site are referred to as HSPP1 and HSPP2 respectively;
(c) identifying the presence of susceptibility to breast carcinomas greatest if that individual is homozygous for the polymorphic risk version of the gene at the 1267 site (P2/P2).

6. A method of managing and treating patients with a predisposition to breast carcinoma, comprising determining whether the individual possesses a polymorphism in an MHC gene associated with breast carcinoma, wherein the management and treatment of such patient having such polymorphism are promptly treated and managed as patients having a predisposition to breast carcinoma.

7. The method of claim 6 wherein the polymorphism is located on the TNF-α gene at the −308 locus.

8. The method of claim 6 wherein the polymorphism is located on the HSP70-2 gene at the 1267 locus.

9. A method of screening to identify compounds which stimulate or inhibit the synthesis or action of a polymorphism in an MHC gene associated with breast carcinoma, comprising screening compounds with desired polymorphism sites and identifying those compounds which act as agonists toward the sites and those compounds which inhibit activity as antagonists.

10. The method of claim 9 wherein the polymorphism site is selected from the group consisting of TNF-α and HSP70-2.

11. A agonist or antagonist identified by the method of claim 10.

12. A method of treating patients comprising identifying a patient with a predisposition to breast carcinoma by identifying polymorphisms in an MHC gene associated with breast carcinoma and administering to such patient an effective amount of an antagonist identified in claim 11 in a pharmaceutically acceptable carrier.

13. A method of predicting the clinical outcome of a breast carcinoma patient comprising determining whether the individual possesses a TNF2 homozygous genotype of the TNF-α gene at the −308 locus wherein the TNF-breast carcinoma-specific overall survival and disease-free survival are considered to be shortest in patients carrying the TNF2 homozygous genotype.

14. A method of predicting the clinical outcome of a breast carcinoma patient comprising determining whether the individual possesses a HSP70-2 homozygous genotype, wherein the survival rate is the longest in the group of breast-carcinoma patients carrying the HSP-P2 homozygous genotype.
